(19) United States
(12) Patent Application Publication
(10) Pub. No.: US 2012/0041388 A1
(43) Pub. Date: Feb. 16, 2012

(54) SYRINGE HANDLE DEVICE

(75) Inventor: Eric N. Blomquist, Scottsdale, AZ (US)

(73) Assignee: Farnam Companies, Inc., Phoenix, AZ (US)

(21) Appl. No.: 12/854,729

(22) Filed: Aug. 11, 2010

(51) Int. Cl. A61M 5/315; A61D 7/00

(52) U.S. Cl. 604/227

(57) ABSTRACT

A syringe handle that is separately formed from a syringe and enabled to be later attached to a syringe is presented along with methods for using the handle. In some embodiments, handles can be attached and then detached from the syringes for re-use. Handles can be ergonomically elongated so as to not only support a user's index and middle fingers, but also his or her ring finger and/or pinkie as well.

### CROSS-REFERENCES TO RELATED APPLICATIONS

NOT APPLICABLE

### BACKGROUND

1. Field of the Invention

Embodiments of the present invention generally relate to means for introducing material to the body for therapeutic purposes, specifically an ergonomic, discrete handle that can be attached to and, in some embodiments, detached from a syringe.

2. Description of the Related Art

Syringes are used for a wide variety of applications and are especially common in the medical and veterinary fields. In medical applications, syringes are most often associated with the hypodermic administration of injections into the body using a narrow, hollow metal needle. In veterinary applications, syringes are also used for applying medicines into the mouths of animals as well as into other orifices. These syringes often employ plastic tubing with a nozzle instead of a metal needle.

As with some human patients, animals can be uncooperative when undergoing treatment. An animal can be restless and fidgeting when held captive for examination, or it can be downright nippy and hostile. Many animals regard their mouths as personal areas and do not appreciate being prodded or poked there, especially by a human stranger. Many animals instinctively shimmy or back away from someone touching a sore, inflamed, or other sensitive area.

A large animal, such as a horse, can be dangerous to a treating veterinarian because it is larger and physically more powerful than a human. For example, a horse can chomp down on a veterinarian's fingers if they reach within its mouth. The use of a syringe for oral administrations of drugs can help keep the veterinarian's fingers away from the bite of the animal.

Many syringes have a tube or barrel with an integrated flange at one end. The integrated flange acts as a handle for the user's fingers. A user holds the syringe with an index finger resting on the flange on one side of the barrel, a middle finger resting on the flange on an opposite side of the barrel, and a thumb resting on the plunger. The user operates the syringe by pressing down on the plunger with his or her thumb while exerting opposite force with his or her index and middle fingers, essentially drawing the thumb and fingers toward each other. This forces the plunger through the barrel, which in turn squeezes fluid from the syringe nozzle.

It has been found that some viscous liquids and pastes require a great deal of thumb force to dispense from a syringe, so much so that some people lack the hand strength to dispense them. Still others strain their hands when exerting such force on a syringe and gradually lose precise control over the location of the distal tip of the syringe.

One solution in the equine industry has been to extend one side of the integrated syringe flange so that a user can rest her ring finger on the integrated flange. An example of such a product is the Sure-Grip™ Syringe for IverCare® antiparasitic preparations, produced by Farnam Companies, Inc., the assignee of the present invention. Such syringes help safely and accurately deliver oral medicines and supplements to horses.

### BRIEF SUMMARY

Devices, methods, and systems of the present disclosure relate to a discrete handle that can be attached to a syringe to allow a user to better grip the syringe. In some embodiments, the handle can be detached from the syringe and attached to other syringes. This can enable some users to carry his or her own custom handle for dealing with syringes. There can be a carved area out in the handle in which the small flange of a syringe can be recessed.

The handle can have a longer side (opposite the index finger side) for resting not only a middle finger, but also for ergonomically resting a ring finger and/or pinkie as well. The handle can be made of one-piece, injection molded plastic with strengthening ribs running laterally along the edges.

Syringe and handle assemblies are also described in the present disclosure as well as methods for using the syringe and handle.

An embodiment relates to an ergonomic handle device for a syringe, comprising a handle body, the body having a lateral aperture therethrough, thereby forming a first end of the elongated body and, opposite the aperture, a second end of the elongated body. The handle device further comprises an index finger support region at the first end of the elongated body, the index finger support region adapted to receive an index finger of an operator, a syringe attachment region on the body adapted to attach the body to a syringe, and an other-finger support region at the second end of the elongated body, the other-finger support region adapted to receive at least one of a middle finger, a ring finger, and a pinkie of the operator. The embodiment allows an operator to firmly grasp an assembly of the handle device and a syringe by resting an index finger on the index finger support region, at least one other finger on the other-finger support region, and a thumb on a plunger of the syringe.

An embodiment relates to an ergonomic handle device for a syringe, comprising an elongated handle body, the body having a lateral aperture therethrough and off center from a longitudinal center of the elongated body, thereby forming a short end of the elongated body and, opposite the aperture, a long end of the elongated body. The handle device further comprises an index finger support region at the short end of the elongated body, the index finger support region adapted to receive an index finger of an operator, a syringe attachment region on the body adapted to attach the body to a syringe, and a multi-finger support region at the long end of the elongated body, the multi-finger support region adapted to receive at least two of a middle finger, a ring finger, and a pinkie of the operator. The embodiment allows an operator to firmly grasp an assembly of the handle device and a syringe by resting an index finger on the index finger support region, at least two other fingers on the multi-finger support region, and a thumb on a plunger of the syringe.

An embodiment relates to a handle and syringe assembly, comprising an elongated handle body, having a index finger support region, an aperture adjacent the index support region, and a multi-finger support region opposite the hole from the index finger support region. The handle and syringe assembly further comprise a syringe extending through the aperture and releasably coupled to the handle body and an intravenous, viscous paste within the syringe. In the embodiment, the multi-finger support region allows an operator more control to inject the viscous paste into a restless, fidgeting animal.

An embodiment relates to a method, comprising coupling a handle device to a syringe to form an assembly, the handle device having an elongated body with a lateral aperture therethrough and off center from a longitudinal center of the elongated body, thereby forming a short end of the elongated body and, opposite the aperture, a long end of the elongated body. The method further comprises grasping the assembly by placing an index finger on the short end, at least two of a middle finger, a ring finger, and pinkie on the long end, and a thumb on a plunger of the syringe, inserting a needle of the syringe into an animal or human, depressing the plunger by squeezing the thumb on the plunger and using the index finger and at least two other fingers for support, and detaching the handle device from the syringe, thereby allowing for re-use of the handle device on another syringe. The method can optionally further comprise coupling the handle device to another syringe and can include using gloved hands.

An embodiment relates to a handle device for a syringe, comprising a handle body, the handle body having an index finger support region adapted to receive an index finger of a user, an other-finger support region adapted to receive at least one of a middle finger, ring finger, and pinkie of the same hand as the index finger, and a syringe attachment area between the finger support regions, the syringe attachment region adapted to be attached by the user to a syringe.

An embodiment relates to a method, comprising holding a handle device by placing an index finger on an index finger support region of the handle device, and at least one of a middle finger, ring finger, and pinkie of the same hand as the index finger on an other-finger support region of the handle device. After holding the handle device as described, the method further includes attaching a syringe to a syringe attachment region adapted to be attached by a user to the syringe. The attachment region is on the handle device between the index finger support region and other-finger support regions. The method further includes placing a thumb of the same hand as the index finger on a plunger of the syringe and depressing the plunger with the thumb, using the index finger and the at least of the middle finger, ring finger, and pinkie for support. The method can optionally include detaching the handle device from the syringe, thereby allowing for re-use of the handle device on another syringe.

### BRIEF DESCRIPTION OF THE DRAWINGS

FIG. 1 is an axonometric view of a top of a handle in accordance with an embodiment.

FIG. 2 is an axonometric view of a bottom of a handle of FIG. 1.

FIG. 3 is an axonometric view of a syringe connected with a handle in accordance with an embodiment.

FIG. 4A is another axonometric view of the syringe connected with the handle of FIG. 3.

FIG. 4B illustrates a hand gripping the syringe/handle assembly of FIG. 4A.

FIG. 5 is a top view of the handle of FIG. 1.

FIG. 6A is a cross section of the handle of FIG. 5.

FIG. 6B is a detail view of FIG. 6A.

FIG. 7 is a side view of the handle of FIG. 1.

FIG. 8 is an end view of the handle of FIG. 1.

FIG. 9 is a flow chart of a process in accordance with an embodiment.

### DETAILED DESCRIPTION

The present disclosure relates to syringe handles that are formed separately from syringes and then attached to syringes to allow users to better grip the syringes with their fingers and depress the syringes' plungers. Optionally, the handles can be detached from the syringes and attached to other syringes. A recessed portion in the handle can interface with the flange of a syringe such that the flange snaps into the recess.

The syringe handles can have longer ends, opposite an index finger end, for resting not only a middle finger, but also a ring finger and/or pinkie finger. The handles can be manufactured in one piece from thermoplastic for inexpensive mass production.

Prior art syringes often have integrated flanges which serve as handles. The inventor of the present invention recognized a need in the art for handles to be manufactured as separate pieces from syringes and attached to the syringes later. One of the technical advantages of this is that as new handle designs are created, syringe manufacturing machines do not need to incorporate new molds. Older syringes can be used with the new handles. This saves time, labor, and money, and offers manufacturing convenience. It also offers manufacturing flexibility in that handles can be added to syringes later as the market demands. Furthermore, in certain industries with lower volumes (e.g., the equine industry), low cost syringes that were perfected, tested, and mass-produced for industries with higher volumes (e.g., the health care industry) can be used with handle features that are better suited for the lower volume industry. For example, a larger handle may be preferred when dealing with large animals than with people. A larger handle may even be necessary to dispense heavy pastes and other viscous fluids that are common in the economic animal and equine industries.

Producing a separate handle also offers packaging flexibility because the packaging can be smaller. A long, integrated handle protrudes laterally from syringe, forming a ‘+’ or ‘T’ with the syringe. This takes up packaging space. A separate handle can be packaged parallel to the syringe. The parallel syringe and handle may be packaged closer together than the ‘+’ or ‘T’ configuration, saving cardboard carton and/or blister packaging space.

Not only can different handles be used in different industries with the same model of syringe, but a separate handle allows one to decide whether to use a handle at all depending on a specific patient. For example, a handle can be used for particularly feisty animals that might give the veterinarian trouble. No handle may be used for cases in which the animal is so feeble or docile that it does not pose trouble to the veterinarian.

Handles may or may not be used depending on the specific operator. Elongated handles that have enough room to rest one's middle finger, ring finger, and/or pinkie finger can help those with weaker hands by providing more leverage. Operators with weaker or compromised hands may elect to use a large, ergonomic handle on their syringes. For example, ranchers with fatigued, arthritic, or temporarily weakened hands may elect to use the larger handles at the end of their shift.

Different handles can be used depending on the specific operator. A person with a hand disability may carry around his or her own, custom-fit handle. The custom-fit handle may be curved and optimized to conform to his or her thumb and finger lengths, strengths, and gripping style, and pulling mechanics. People can carry these handles around on their key chains like church keys.

Yet another technical advantage of some of the embodiments is that handles can be re-used. In re-use, there is less waste, including that of plastics and petroleum products. If handles are bought and sold separately, then there can be less shipping weight for syringes that would otherwise have an integrated handle.

Although many of the examples herein refer to syringes for animals, embodiments also apply to syringes for humans, such as in a hospital setting. Furthermore, syringes for other industries are envisioned, including those for caulking guns and glues and wide-barreled, ‘big-bore’ syringes and applicators. The term “syringe” generally includes a piston pump in a tube that can expel a fluid (e.g., aerosol, colloid, emulsion, foam, gel, suspension, pure liquid or gas) through an orifice at an end of the tube.

In the prior art, handles were integrated with syringes barrels as one piece. This may have been seen as easy to manufacturer. No ‘snap on’ or other attachment means need to be engineered because the parts are formed together. Separate parts put together are often weaker than parts that are formed integrally, such as parts that are formed in the same thermoplastic mold. Strength may be deemed critical for syringes and medical devices.

Previously, separate parts may have been seen as being sterile. Parts that are formed separately and joined later often include small gaps between them where they do not fit together perfectly. These gaps can pick up dirt and other contaminates, therefore impairing the sterility of a device. Separate parts, therefore, may be considered less ideal for a medical or veterinary application.

One does not normally re-use medical devices, such as syringe parts, because of the fear of contamination. However, the design of some embodiments of the present disclosure keeps the junction between parts away from critical areas. A recess portion for a syringe flange can help keeps the syringe from rotating and better secure it to the handle, using the whole flange as a bearing surface. The handle can even help keep a syringe's flange, which may include sharp plastic edges and mold lines, out of the way from catching on fur or hair of an animal.

The figures will now be used to illustrate different embodiments in accordance with the invention. The figures are specific examples of embodiments and should not be interpreted as limiting embodiments, but rather exemplary forms and procedures.

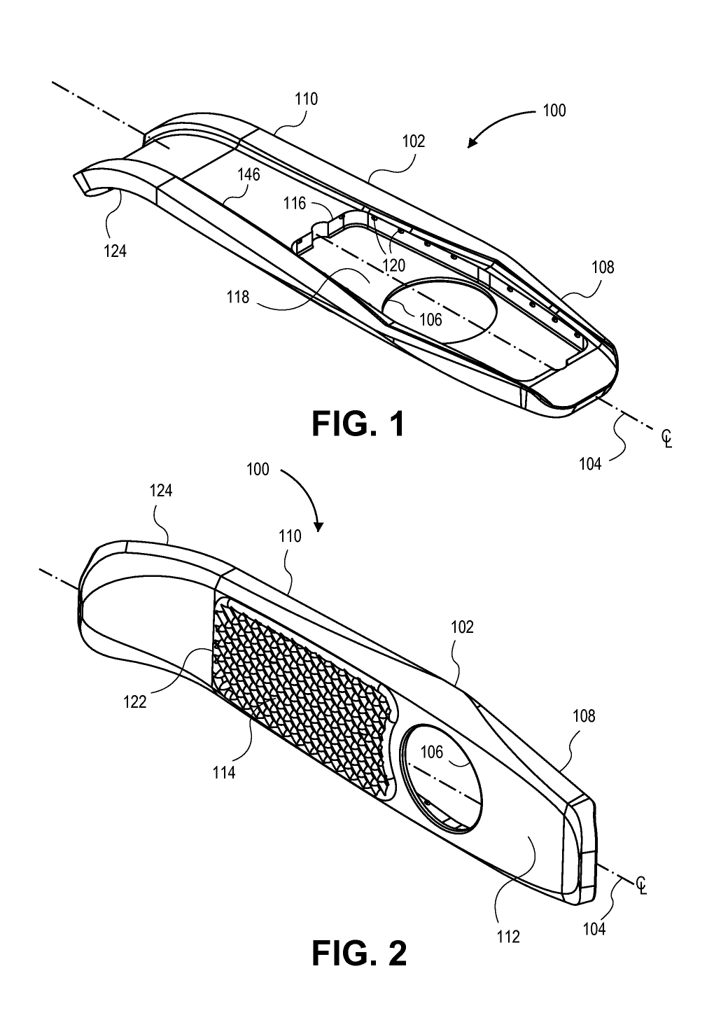

FIGS. 1 and 2 are axonometric views of a top and bottom of a handle, respectively, in accordance with an embodiment. Handle device 100 includes elongated handle body 102 having longitudinal axis 104. In the exemplary embodiment, longitudinal axis 104 is along a center plane of the handle. Handle device is symmetric to the left and the right of the center plane. Handle body 102 includes aperture 106. Aperture 106 is a through hole that extends laterally up and down through handle body 102. Aperture 106 divides handle body 102 into short end 108 and long end 110, the ends being on opposite sides of the hole from one another.

FIG. 1, which shows the top, proximate side of handle body 102, shows syringe attachment region 116 that is adapted to attach the handle body to a syringe. Attachment region 116 includes recessed region 118, which can receive a flange/handle of a syringe. Bumps 120 protrude into recessed region 116 enough to allow the flange of a syringe to be snapped into the recess but not enough so that the syringe cannot be snapped out. Bumps 120 surround the top of the recesses area and ensure a snug fit of the syringe.

Different methods of attachment of the handle to a syringe can be used. For example, a handle can have a strap, hook and loop fasteners (e.g., Velcro®), snaps, bayonet connectors, pin and recess connections, screw(s)/bolt(s), and other releasable means for attachment. Irreversible means of attachment can include adhesives, rivets, one-way snaps, pressure-fit connections, etc.

Ribs 146 extend longitudinally along the outer lateral edges of handle body 102. Ribs 146 provide strength and rigidity to the handle.

FIG. 2, which shows the bottom, distal side of handle body 102, shows index finger support region 112, which has a flattened surface. Index support region 112 can receive force from a index finger. The figure also shows multi-finger support region 114, which has flattened anti-slip treatment 122. Multi-finger support region 114 is long enough for an adult to place a middle finger, ring finger, and/or pinkie (i.e., other fingers than the previously mentioned index finger), and the multi-finger support region can receive force from the fingers alone or in combination.

While wearing gloves, a user may find that a long handle, such as that shown in the figures, may be easier to grip and control than a short handle. In some instances, it may be easier for a gloved operator to place the handle (only) in his or her hand, supported by his index and other finger(s) as if he were going to use it, and then use his other hand to snap a syringe into the handle, although in other instances snapping the handle and syringe together and then gripping the assembly for use might be easier.

Near the tip of long end 110, curved region 124 of multi-finger support region 114 curves upward to form a slope. This slope can meet a user's pinky and otherwise conform to a user's hand.

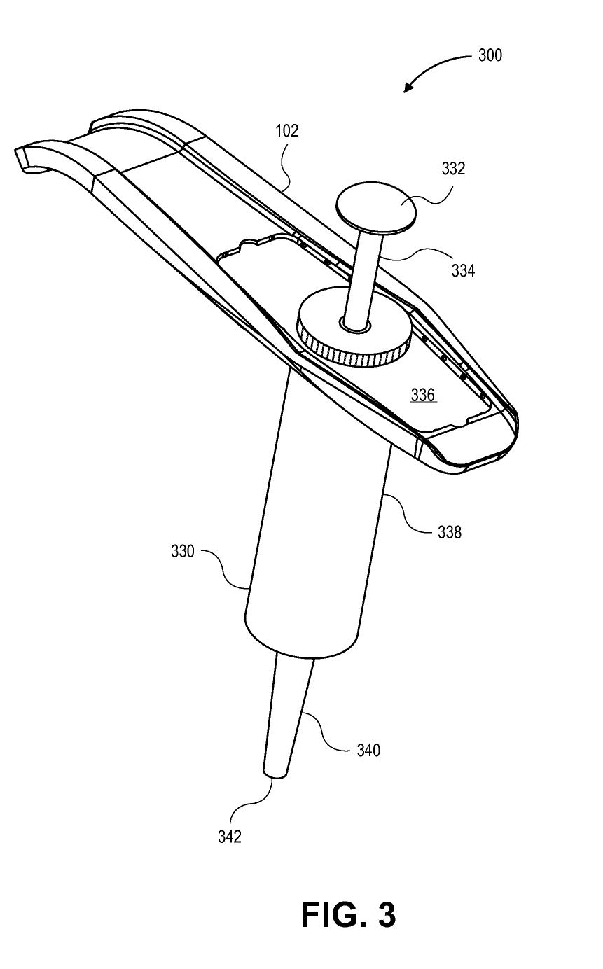

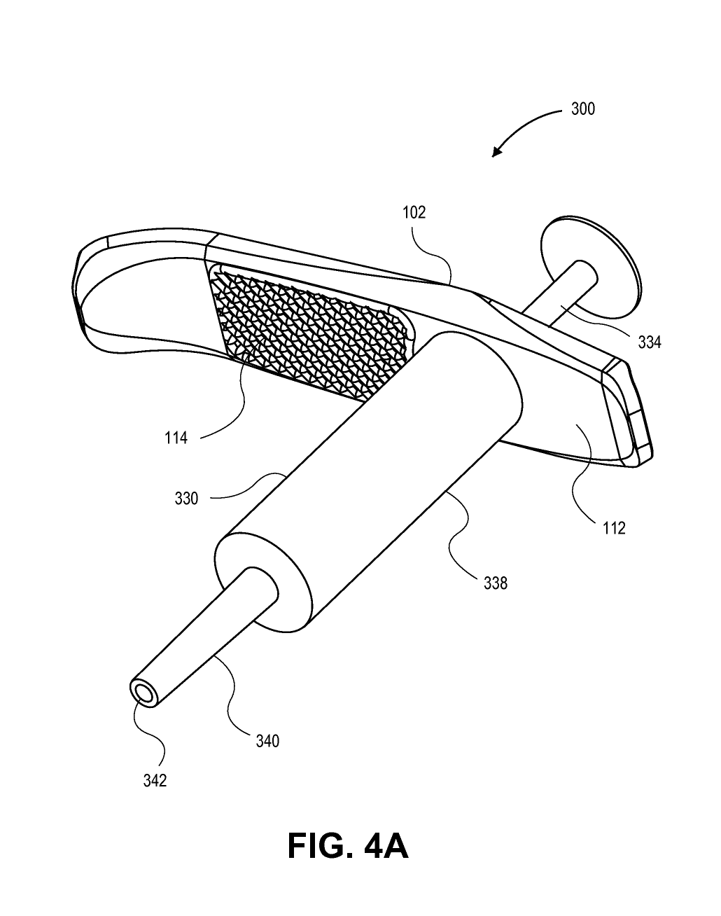

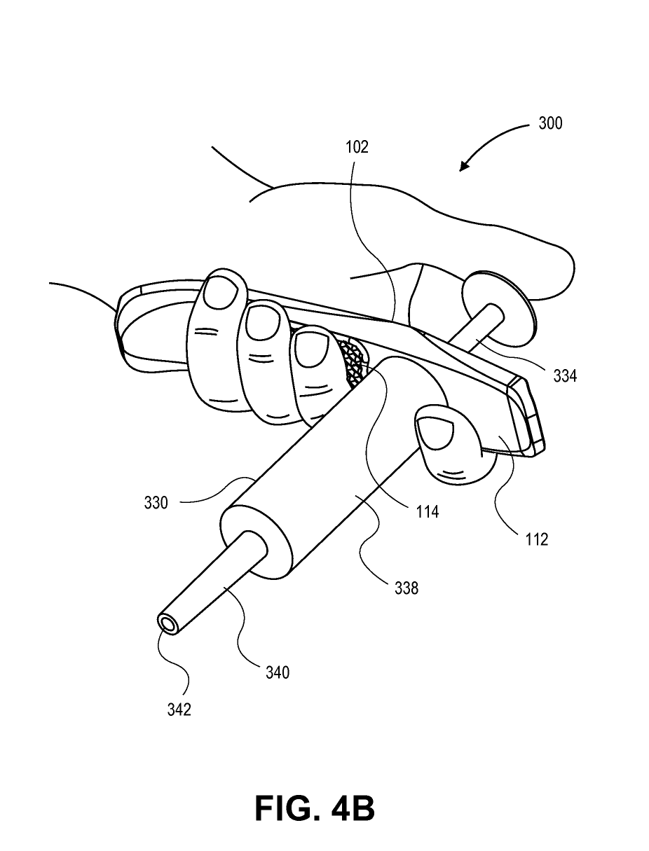

FIGS. 3, 4A, and 4B are axonometric views of a syringe connected with a handle (top and bottom, respectively) in accordance with an embodiment. Handle body 102 is snapped onto syringe 330. Syringe 330 has thumb rest 332 on plunger 334, flange 336, barrel 338, and nozzle/needle 340. To operate the assembly (as shown in FIG. 4B), an operator places his index finger on index finger support region 112, his or her middle finger, ring finger, and/or pinky finger on multi-finger support region 114, and squeezes his or her thumb down on plunger 334. Fluid, such as a liquid, gel, paste, etc., is pushed by a piston within barrel 338 through nozzle/needle 340 out tip 342.

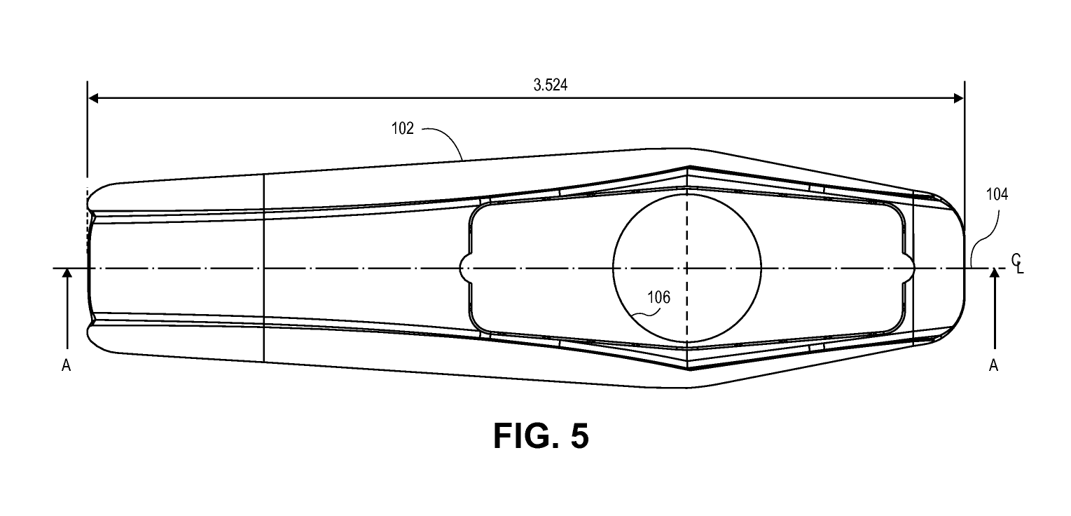

FIG. 5 is a top view of the handle of FIG. 1. A handle body planform longitudinal length of 3.524 inches (8.95 centimeters) with aperture 106 off center about a third as shown has been found to be a comfortable handle. As noted before, handle body 102 is symmetric about a plane through centerline 104.

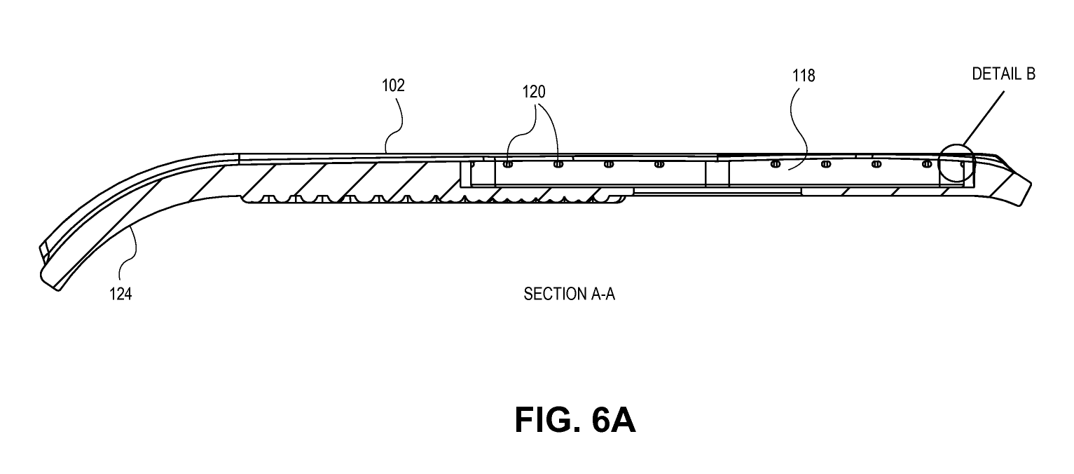

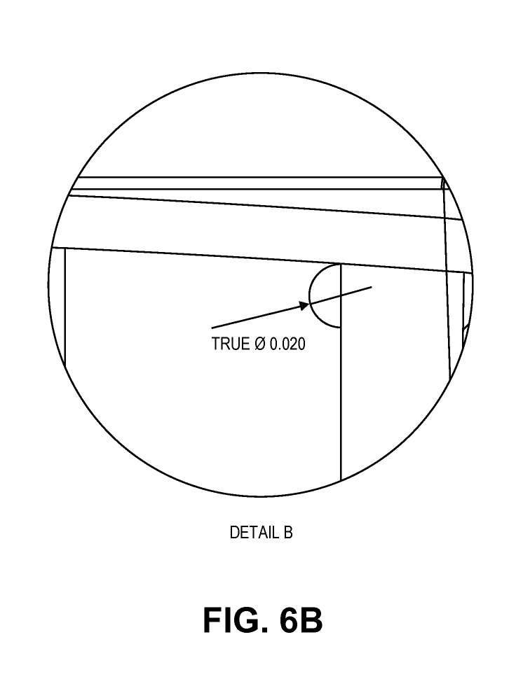

FIG. 6A is cross section A-A of the handle of FIG. 5. Handle body 102 has recessed region 118 extending more than halfway through its thickness. Bumps 120 flank the entrance to recessed region 118. Curved region 124 slopes upward toward the distal face of the handle. This is also toward the needle end of a syringe, if a syringe were attached.

FIG. 6B is a detail view of FIG. 6A. A diameter of 0.020 inches for hemispheric plastic bump 120 has been found to be sufficient to releasably fasten a syringe flange to the handle.

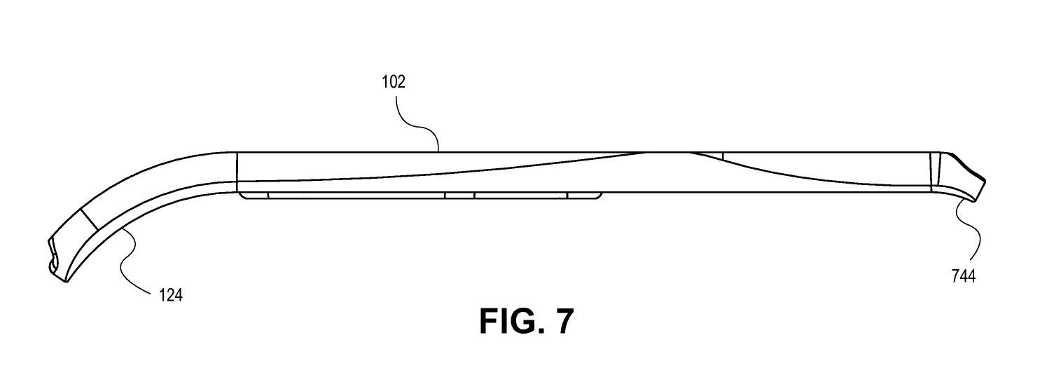

FIG. 7 is a side view of the handle of FIG. 1. Handle body 102 maintains an even overall thickness throughout its width; however, variable thicknesses can be used. The handle has a slightly upturned nose 744 that can help keep one's index finger on the grip.

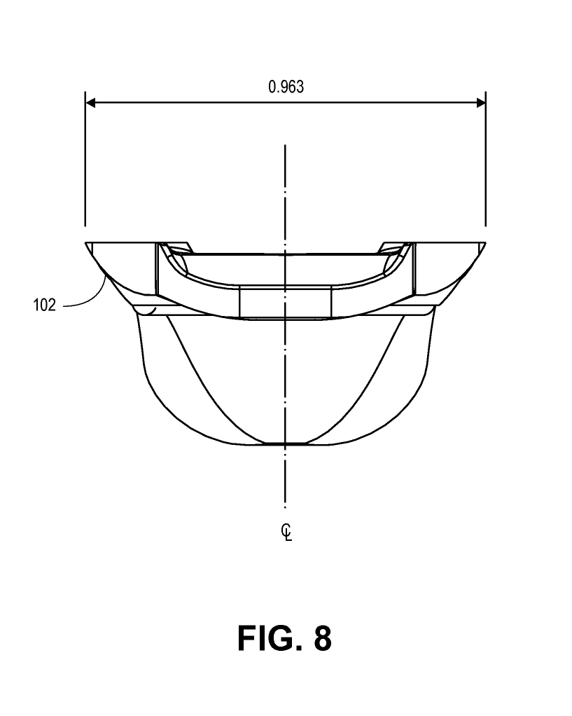

FIG. 8 is an end view of the handle of FIG. 1. A width of 0.963 inches (2.45 centimeters) has been found to be a comfortable width for the handle. Handle body 102 can be injection molded in one piece as a single piece of metal, plastic, or other suitable material. This handle can be much easier on the hand and ergonomic than a tiny, sharp flange of a mass-produced syringe.

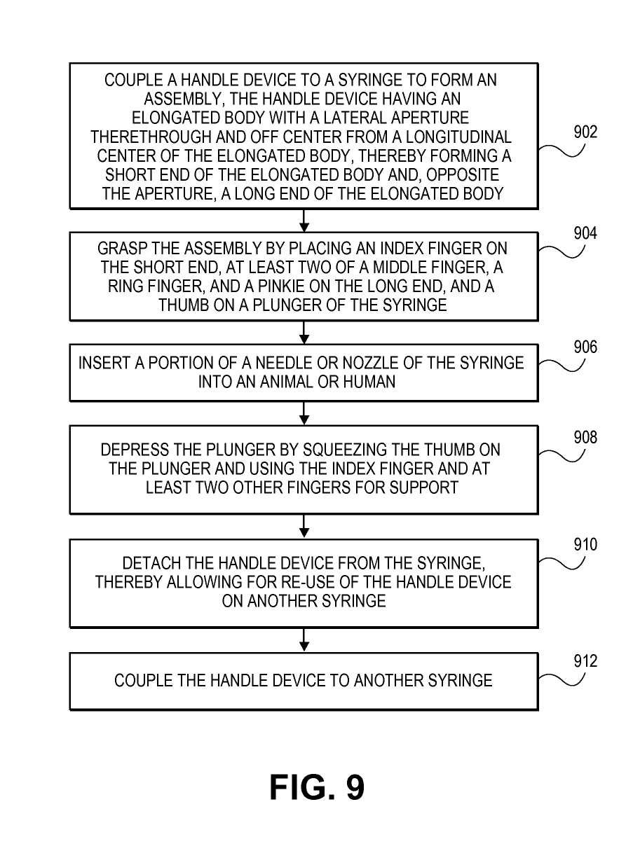

FIG. 9 is a flow chart of a process in accordance with an embodiment. The process can be performed by a doctor, veterinarian, or any other person. In operation 902, a handle device is coupled to a syringe to form an assembly, the handle device having an elongated body with a lateral aperture through it and off center from a longitudinal center of the elongated body, thereby forming a short end of the elongated body and, opposite the aperture, a long end of the elongated body. In operation 904, the assembly is grasped by placing an index finger on the short end, at least two of a middle finger, a ring finger, and a pinkie (finger) on the long end, and a thumb on a plunger of the syringe. In operation 906, a (portion of a) needle or nozzle of the syringe is inserted into an animal or human patient. In operation 908, the plunger is depressed by squeezing the thumb on the plunger while using the index finger and at least two other fingers for support. In operation 910, the handle device is detached from the syringe. This allows for re-use of the handle device on another syringe. In operation 912, the handle device is coupled to another, second syringe. Operations 902-910 can be repeated using the second syringe.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive.

What is claimed is:

1. A handle device for a syringe, comprising:
a handle body, the body having a lateral aperture therethrough, thereby forming a first end of the handle body and, opposite the aperture, a second end of the handle body;
an index finger support region at the first end of the handle body, the index finger support region adapted to receive an index finger of an operator;
a syringe attachment region on the handle body adapted to attach the handle body to a syringe; and
an other-finger support region at the second end of the handle body, the other-finger support region adapted to receive at least one of a middle finger, a ring finger, and a pinkie of the operator,
whereby an operator can firmly grasp an assembly of the handle device and a syringe by resting an index finger on the index finger support region, at least one other finger on the other-finger support region, and a thumb on a plunger of the syringe.

2. The device of claim 1 wherein the syringe attachment region is adapted to releasably attach the handle body to a syringe, thereby allowing the handle body to be re-used with other syringes.

3. The device of claim 1 further comprising:
a recessed region at the attachment region, the recessed region adapted to receive a flange of a syringe.

4. The device of claim 3 wherein the recessed region includes protrusions adapted to secure a flange of a syringe within the recessed region.

5. The device of claim 3 wherein the recessed region is on a side of the handle body opposite the finger support regions.

6. The device of claim 3 wherein the recessed region surrounds the aperture.

7. The device of claim 1 wherein the multi-finger support region comprises a curved region that curves upward toward a needle or nozzle end of an attached syringe.

8. The device of claim 1 wherein the handle body is a single piece of injection-molded plastic.

9. A syringe and handle assembly, comprising:
the handle device of claim 1;
a syringe releasably coupled to the handle device,
whereby the handle device can be decoupled from the syringe and re-coupled to another syringe.

10. The device of claim 1 wherein the lateral aperture is through a longitudinal center of the handle body.

11. A method, comprising:
coupling a handle device to a syringe to form an assembly, the handle device having a handle body with a lateral aperture therethrough, thereby forming a first end of the handle body and, opposite the aperture, a second end of the handle body;
grasping the assembly by placing an index finger on the first end, at least one of a middle finger, a ring finger, and pinkie on the second end, and a thumb on a plunger of the syringe;
depressing the plunger by squeezing the thumb on the plunger and using the index finger and at least one other finger for support; and
decoupling the handle device from the syringe, thereby allowing for re-use of the handle device on another syringe.

12. The method of claim 11 further comprising:
coupling the handle device to another syringe.

13. The method of claim 11 further comprising:
inserting or directing a portion of a needle or nozzle of the syringe into or onto an animal or human.

14. The method of claim 11 wherein the coupling, grasping, depressing, and decoupling operations are performed in the order shown and with a gloved hand.

15. An ergonomic handle device for a syringe, comprising:
an elongated handle body, the body having a lateral aperture therethrough and off center from a longitudinal center of the elongated handle body, thereby forming a short end of the elongated handle body and, opposite the aperture, a long end of the elongated handle body;
an index finger support region at the short end of the elongated handle body, the index finger support region adapted to receive an index finger of an operator;
a syringe attachment region on the elongated handle body adapted to attach the elongated handle body to a syringe; and
a multi-finger support region at the long end of the elongated handle body, the multi-finger support region adapted to receive at least two of a middle finger, a ring finger, and a pinkie of the operator,
whereby an operator can firmly grasp an assembly of the handle device and a syringe by resting an index finger on the index finger support region, at least two other fingers on the multi-finger support region, and a thumb on a plunger of the syringe.

16. The device of claim 15 wherein the syringe attachment region is adapted to releasably attach the elongated handle body to a syringe, thereby allowing the elongated handle body to be re-used with other syringes.

17. The device of claim 15 further comprising:
a recessed region at the attachment region, the recessed region adapted to receive a flange of a syringe.

18. The device of claim 17 wherein the recessed region includes protrusions adapted to secure a flange of a syringe within the recessed region.

19. The device of claim 17 wherein the recessed region is on a side of the elongated handle body opposite the finger support regions.

20. The device of claim 17 wherein the recessed region surrounds the aperture.

21. The device of claim 15 wherein the multi-finger support region comprises a curved region that curves upward toward a needle or nozzle end of an attached syringe.

22. The device of claim 15 wherein the elongated handle body is a single piece of injection-molded plastic.

23. The device of claim 15 wherein the finger support regions have flattened surfaces.

24. The device of claim 15 wherein the elongated handle body is symmetric about a longitudinal plane of the elongated handle body.

25. The device of claim 15 further comprising:
ribs extending longitudinally along lateral edges of the elongated handle body.

26. A syringe and handle assembly, comprising:
the handle device of claim 15;
a syringe releasably coupled to the handle device,
whereby the handle device can be decoupled from the syringe and re-coupled to another syringe.

27. A handle and syringe assembly, comprising:
an elongated handle body, having a index finger support region, an aperture adjacent the index support region, and a multi-finger support region opposite the hole from the index finger support region;
a syringe extending through the aperture and releasably coupled to the handle body; and
an intravenous, viscous paste within the syringe,
whereby the multi-finger support region allows an operator more control to inject the viscous paste into a restless, fidgeting animal.

28. A method, comprising:
coupling a handle device to a syringe to form an assembly, the handle device having an elongated body with a lateral aperture therethrough and off center from a longitudinal center of the elongated body, thereby forming a short end of the elongated body and, opposite the aperture, a long end of the elongated body;
grasping the assembly by placing an index finger on the short end, at least two of a middle finger, a ring finger, and pinkie on the long end, and a thumb on a plunger of the syringe;
depressing the plunger by squeezing the thumb on the plunger and using the index finger and at least two other fingers for support; and
decoupling the handle device from the syringe, thereby allowing for re-use of the handle device on another syringe.

29. The method of claim 28 further comprising:
coupling the handle device to another syringe.

30. The method of claim 28 further comprising:
inserting or directing a portion of a needle or nozzle of the syringe into or onto an animal or human.

31. The method of claim 28 wherein the coupling, grasping, inserting, depressing, and decoupling operations are performed in the order shown and with a gloved hand.
